(19) United States
(12) Patent Application Publication
(10) Pub. No.: US 2024/0226597 A1
(43) Pub. Date: Jul. 11, 2024

(54) LASER SYSTEM FOR ENHANCING REMINERALIZATION AND STRENGTH OF HARD TISSUE

(71) Applicant: Convergent Dental, Inc., Waltham, MA (US)

(72) Inventors: Charles Kerbage, Arlington, MA (US); Ali Badreddine, Boston, MA (US); Stephen Couitt, Auburndale, MA (US); Roni Cantor-Balan, Natick, MA (US)

(21) Appl. No.: 18/413,943

(22) Filed: Jan. 16, 2024

Related U.S. Application Data
(63) Continuation of application No. 17/139,208, filed on Dec. 31, 2020, now Pat. No. 11,918,824.
(60) Provisional application No. 62/956,862, filed on Jan. 3, 2020.

(51) Int. Cl. A61N 5/06; A61C 19/06; A61N 5/067

(52) U.S. Cl. CPC A61N 5/0603; A61C 19/063; A61N 2005/0606; A61N 5/067

(57) ABSTRACT

A laser-based system and method for treatment of hard tissue enhances remineralization and fluoride uptake to improve resistance to demineralization. The system can include a laser source, an optic, and a controller for controlling the laser source and/or the optic to deliver radiation to a treatment region with desirable characteristics (e.g., to remove carbonate without damaging the tissue surface). In some cases, the system includes a handpiece having an optical element (e.g., a lens) mounted within a replaceable cartridge and adapted to modulate a laser beam to render the beam non-ablative, prior to the laser beam's delivery to the treatment region. In some embodiments, treatment with the laser can be combined with a fluoride treatment for enhanced therapeutic effect.

### CROSS-REFERENCE TO RELATED APPLICATIONS

This application is a continuation of U.S. application Ser. No. 17/139,208, filed Dec. 31, 2020, which claims the benefit of priority to U.S. Provisional Patent Application No. 62/956,862 entitled “Laser System for Enhancing Remineralization and Strength of Hard Tissue,” filed on Jan. 3, 2020, the contents of which are incorporated by reference herein in their entirety.

### TECHNICAL FIELD

The present invention generally relates to the treatment of hard tissue using a light-emitting device (e.g., a laser source) and, more particularly, to enhancing remineralization and strength of hard tissue by directing radiation emitted by a laser source to the hard tissue.

### BACKGROUND

Dental caries is a disease where tooth mineral is dissolved by acid and, if it progresses, cavities are formed. Specific bacteria in the mouth form organic acids when they feed on fermentable carbohydrates, such as sugar. These organic acids readily dissolve the carbonated hydroxyapatite mineral of tooth enamel causing mineral loss (demineralization) that initially shows up as a non-cavitated “white spot” that eventually becomes a cavity if the process continues.

The mineral that comprises about 95% by weight of tooth enamel is often described as being hydroxyapatite, which is a form of calcium phosphate. However, in actuality, tooth enamel contains numerous impurities and inclusions as a result of being formed in a biological fluid system in the human body. The mineral is better described as a carbonated hydroxyapatite in which about 1 in every 10 phosphate groups are replaced by carbonate, making the mineral many-fold more soluble in acid than pure hydroxyapatite, which has no carbonate inclusions.

Research has been performed on the use of lasers for the treatment of caries, e.g., by ablating decayed tissue so that it can be replaced with a filling material. Lasers such as Er: YAG and Nd: YAG lasers are primarily absorbed by water within the tooth rather than hydroxyapatite and may cause undesired damage to the tissue surface during a heating process. This is partly because a significant amount of the water is removed before hydroxyapatite reaches the high temperatures required for the acid-resistant effect and also from a blasting effect, as the water under the surface turns to steam. More recent research has revealed that certain CO₂ laser wavelengths are better absorbed in hydroxyapatite and can be effective in ablating dental tissue.

However, significantly less is known about the use of lasers to prevent caries formation and acid dissolution in the first place. Preliminary academic research has suggested that the enamel surface can be modified by heating it with short laser pulses and that such heating may lead to improved caries resistance. However, significantly more research is needed to determine the desired operating parameters for such laser treatments and to adapt the academic research to a functional system appropriate for commerical use with patients. Additionally, little is know about the effects of such laser treatments in combination with conventional caries resistance treatments, such as fluoride applications.

### SUMMARY

Embodiments of the invention described herein relate to a laser treatment system that can be used to enhance both strength and reminzeralization of hard dental tissue. In some implementations, the invention includes the combined use of a laser treatment system and conventional treatement techniques such as fluoride applications, for enhanced effect. The inventors discovered that laser irradiation of dental enamel that results in the removal of carbonate from hydroxyapatite also improves the absorption of fluoride, resulting in a further modified enamel surface that approaches the composition of the less soluble form of calcium phosphate known as fluorapatite, which is even more resistant to acid attack than hydroxyapatite.

In various embodiments, the invention includes a laser source that operates in the wavelength range of 9-11 μm, such as a CO₂ laser. CO₂ lasers have several advantages over other types hard tissue lasers (e.g., Er: YAG lasers), e.g., the absorption coefficient in hydroxyapatite is about 2 factors higher. The invention can feature a handpiece for directing the 9-11 μm laser beam to a hard tissue surface in the oral cavity with desirable efficiency, minimal technique sensitivity, and a fast treatment time.

The system can be adapted to scan the laser beam using a variety of scanning techniques, e.g., using galvo-mirrors. The laser beam can be scanned across the treatment region using particular pattern(s) to allow for efficient energy delivery, producing enough localized photothermal effect to contract collagen without damaging (burning or charring) the tissue. Some such patterns are described in more detail in U.S. Patent Publication No. 2017/0319277, which is incorporated herein by reference in its entirety and attached as Appendix A.

The system may also include a laser source controller than can adjust one or more treatment parameters (e.g., laser pulse duration) according to the type of treatment selected and/or the type of tissue being treated. Various treatment parameters are described in more detail below and also in U.S. Patent Publication No. 2018/0325622, which is incorporated herein by reference in its entirety and attached as Appendix B.

In general, in one aspect, embodiments of the invention feature a system for treating a hard dental tissue. The system can include a laser source for generating a laser beam, an optic in optical communication with the laser source adapted to direct the laser beam to a treatment surface of the hard dental tissue, and a controller adapted to control the laser source and the optic to deliver the laser beam to the treatment surface to treat an area of the hard dental tissue at a rate in a range from 10 cm²/min to 20 cm²/min with a fluence in a range from 0.4 J/cm² up to 1.2 J/cm² to: (i) remove at least some carbonate from the treatment surface to generate an acid resistant surface without damaging the hard dental tissue, and (ii) reduce a ΔZ value of the hard dental tissue by at least 10% relative to untreated hard dental tissue.

In various embodiments, the laser source can include a CO₂ laser source. The laser beam can include a wavelength in a range from 9 μm to 11 μm. In some instances, the laser beam can have a spot size at the treatment surface in a range from 0.2 mm to 5 mm. The optic can include a galvanometer and/or a turning mirror. In some instances, the controller is further adapted to control the laser source to deliver the laser beam to the treatment surface in a series of pulses. In some cases, each pulse in the series of pulses include a pulse energy in a range from 0.1 mJ to 50 mJ. In some cases, each pulse in the series of pulses includes a pulse duration in a range from 1 μsec to 100 μsec. In some cases, each pulse in the series of pulses includes a repetition rate in a range from 0.05 Hz to 10 Hz. The series of pulses can include a duty cycle in a range from 0.1 to 10.

In some embodiments, the controller is adapted to deliver the series of pulses to the treatment surface in a pattern. The pattern can include a diameter in a range from 1 mm to 5 mm. The pattern can include a number of locations in a range from 1 to 1,000 (e.g., 217 locations). The spacing between each location in the pattern can be in a range from 0.1 mm to 5 mm. In some instances, the controller is also adapted to control the laser source to deliver the laser beam to the treatment surface to, when combined with a fluoride treatment, reduce a ΔA value of the dental tissue by at least 20% relative to hard dental tissue subject to the fluoride treatment. In some cases, the system can also include a fluoride delivery system adapted to deliver the fluoride treatment to the treatment surface. In some instances, the controller is further adapted to control the laser source to deliver the laser beam to the treatment surface to reduce a ΔS value of the dental tissue by at least 68% relative to untreated dental tissue. In some cases, the controller is further adapted to control the laser source to deliver the laser beam to the treatment surface to, when combined with a fluoride treatment, reduce a ΔS value of the dental tissue by at least 18% relative to hard dental tissue subject to the fluoride treatment.

In general, in another aspect, embodiments of the invention relate to a method for treating a hard dental tissue. The method can include the steps of: generating a laser beam using a laser source, directing the laser beam to a treatment surface of the hard dental tissue using an optic in optical communication with the laser source, and controlling the laser source and the optic using a controller to deliver the laser beam to the treatment surface to treat an area of the hard dental tissue at a rate in a range from 10 cm²/min to 20 cm²/min with a fluence in a range from 0.4 J/cm² up to 1.2 J/cm² to: (i) remove at least some carbonate from the treatment surface to generate an acid resistant surface without damaging the hard dental tissue, and (ii) reduce a ΔA value of the hard dental tissue by at least 10% relative to untreated hard dental tissue.

In various embodiments, the laser source includes a CO₂ laser source. The laser beam can include a wavelength in a range from 9 μm to 11 μm. In some instances, the laser beam can have a spot size at the treatment surface in a range from 0.2 mm to 5 mm. The optic can include a galvanometer and/or a turning mirror. In some instances, the controller is further adapted to control the laser source to deliver the laser beam to the treatment surface in a series of pulses. In some cases, each pulse in the series of pulses include a pulse energy in a range from 0.1 mJ to 50 mJ. In some cases, each pulse in the series of pulses includes a pulse duration in a range from 1 μsec to 100 μsec. In some cases, each pulse in the series of pulses includes a repetition rate in a range from 0.05 Hz to 10 Hz. The series of pulses can include a duty cycle in a range from 0.1 to 10.

In some embodiments, the controller is adapted to deliver the series of pulses to the treatment surface in a pattern. The pattern can include a diameter in a range from 1 mm to 5 mm. The pattern can include a number of locations in a range from 1 to 1,000 (e.g., 217 locations). The spacing between each location in the pattern can be in a range from 0.1 mm to 5 mm. In some instances, the controlling step further includes using the controller to deliver the laser beam to the treatment surface to, when combined with a fluoride treatment, reduce a ΔA value of the dental tissue by at least 20% relative to hard dental tissue subject to the fluoride treatment. In some cases, the method can further include the step of delivering the fluoride treatment to the treatment surface. In some instances, the controller is further adapted to control the laser source to deliver the laser beam to the treatment surface to reduce a ΔS value of the dental tissue by at least 68% relative to untreated dental tissue. In some cases, the controlling step further include using the controller to control the laser source to deliver the laser beam to the treatment surface to, when combined with a fluoride treatment, reduce a ΔS value of the dental tissue by at least 18% relative to hard dental tissue subject to the fluoride treatment.

### BRIEF DESCRIPTION OF THE DRAWINGS

In the drawings, like reference characters generally refer to the same parts throughout the different views. In the following description, various embodiments of the present invention are described with reference to the following drawings, in which:

FIG. 1 is a schematic perspective view of a laser treatment system, according to various embodiments;

FIG. 2 is a schematic cross-sectional side view of a handpiece including an optical cartridge, according to various embodiments;

FIG. 3 is an example plot of a beam size diameter vs. distance from the handpiece exit orifice, according to various embodiments;

FIG. 4 is a depiction of an example treatment pattern, according to various embodiments;

FIGS. 5A-D depict lased and unlased tissue surfaces under various treatment conditions, according to various embodiments;

FIG. 6 depicts an experimental setup used to demonstrate performance of the laser treatment system, according to various embodiments;

FIG. 7 depicts another experimental setup used to demonstrate performance of the laser treatment system, according to various embodiments;

FIG. 8A is an example chart showing knoop hardness number measurements of various tissue surfaces subject to different pH cycle tests, according to various embodiments;

FIG. 8B is an example plot depicting changes in hardness of various tissue surfaces subject to different pH cycle tests, according to various embodiments;

FIG. 9 is an example chart showing ΔA measurements of various tissue surfaces subject to treatments at different fluence levels, according to various embodiments;

FIGS. 10A-C are an example charts showing ΔS measurements, surface loss measurements, and surface microhardness measurements, respectively, of various tissue surfaces subject to treatments at different fluence levels, according to various embodiments;

FIGS. 11A-B are graphs showing example ΔA vs. ΔS measurements, according to various embodiments;

FIGS. 12A-C are a table and graphs showing example ΔS and ΔA measurements for various tissue surfaces subject to treatment at different fluence levels, according to various embodiments; and

FIG. 13 is a chart providing example laser and treatment parameter values, according to various embodiments.

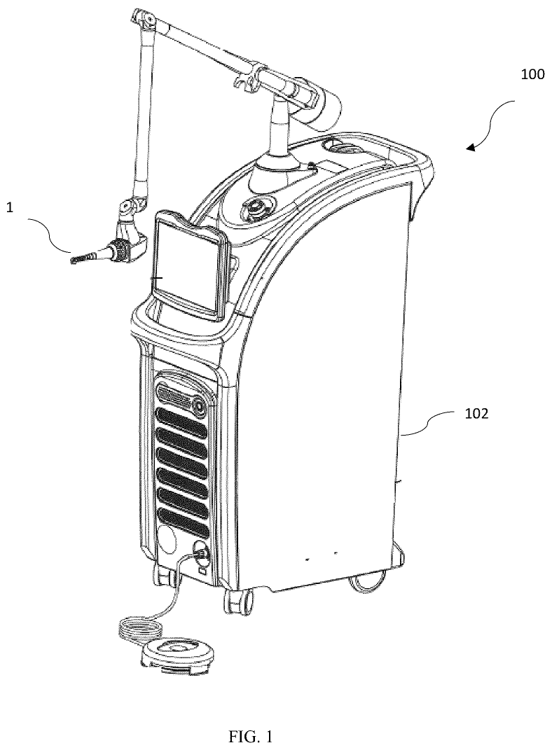

### DETAILED DESCRIPTION

In various embodiments, the present invention is directed to an improved laser treatment system 100. The system 100 can reduce caries formation and acid dissolution of treated hard dental tissue when compared with conventional devices. The system 100 can include a hand piece 1 that delivers laser pulses that heat the hard tissue to remove carbonate impurities from the hydroxyapatite (and thereby enhance remineralization/decrease demineralization) without damaging the tissue. As used herein, the phrase “damaging the tissue” refers to one of burning, charring, or melting of the tissue. The removal of carbonate alone is not considered to be “damaging the tissue,” under the definition used herein. Removal of carbonate without damaging the tissue is further described in U.S. Patent Publication No. 2018/0325622, which is incorporated herein by reference in its entirety and attached as Appendix B.

In various embodiments, the system 100 can also feature (i) a laser beam having a long working range (defined below), (ii) a coolant delivery system for delivering coolant (air, water, mist, etc.) to the oral treatment region and optionally (iii) a fluoride delivery system for applying fluoride to the treatment region. This application will often describe treatment of a hard dental tissue; however, in general, the inventions described herein can be adapted to be used with any suitable hard tissue (e.g., jaw, skull, and other bone regions).

In some embodiments, the laser treatment system 100 includes a CO₂ laser source 102 operating at a wavelength in a range of 9-11 μm (e.g., 9.3 μm) and a handpiece 1 configured to enable uniform treatment of all teeth with minimal change in technique sensitivity. The system 100 can accomplish treatment efficiently, with a fast treatment time and without need for anesthesia.

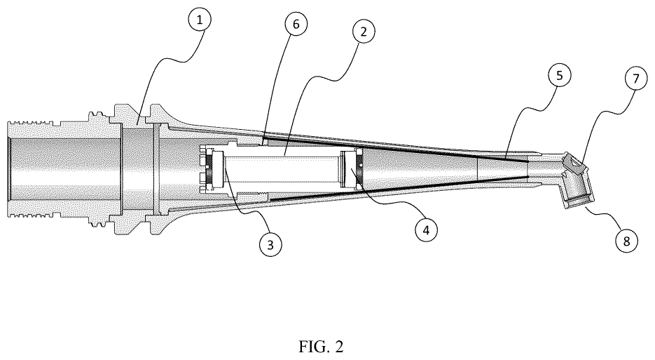

As shown in FIG. 2, the handpiece 1 may be structured and designed to receive an optical cartridge 2. The optical cartridge 2 can include at least one optical lens to modulate a laser beam passing therethrough (e.g., to generate a collimated laser beam). For example, as shown in FIG. 2, the optical cartridge can include an upstream optical lens 3 and a downstream optical lens 4. The optical cartridge 2 can be retained in the handpiece 1 using any known technique, e.g., with threading 6. The ability to replace or remove the optical cartridge 2 allows switching the laser between treatment modes, such as from an ablative mode to a non-ablative treatment mode and vice versa. In addition, the handpiece 1 can include channels or tubing 5 to deliver non-laser substances to the treatment region, e.g., cooling fluids (e.g., air, water, and mist combinations thereof) and/or a fluoride based fluid to enhance acid resitance and improve fluoride uptake. The handpiece can include an exit orifice 8 out of which the laser beam is directed, in some embodiments via a turning mirror 7. In some embodiments, the laser beam can be scanned through the hand piece and across a treatment region using galvanometers disposed upstream of the optical cartridge 2.

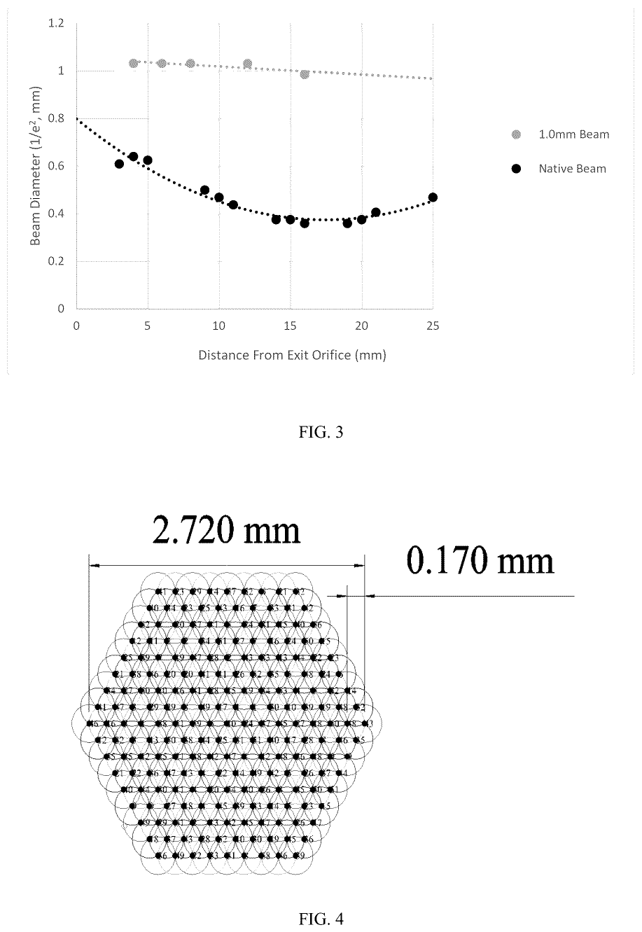

In some embodiments, the optical cartridge 2 can provide a laser beam having a relatively long working range. FIG. 3 is a plot of beam diameter (1/e²) vs. distance from the exit orifice 8 of the handpiece 1, for a 1.0 mm beam generated with the optical cartridge 2 and for a native beam generated without the optical cartridge 2. The example data demonstrates the capability of the optical cartridge to produce a collimated and larger beam size (1 mm) than the original beam size (0.4 mm) and uniform over long working range (e.g., 5-20 mm). Because fluence is a measure of energy per unit area, generating a collimated beam and maintaining a uniform beam area (spot size) along the length of the laser beam (as opposed to a non-collimated beam that converges and diverges about a focal point) can generate a laser beam having a lower fluence which, in some instances, may result in the laser beam being non-ablative. The larger spot size can also enable for more surface area of the treatment region (e.g., a tooth) to be covered more quickly, thereby reducing treatment time.

In various embodiments, the laser is pulsed and scanned according to certain patterns to achieved desired levels of carbonate removal. Moreover, the laser source may be spatially scanned to provide different pulse energies at different locations as shown in FIG. 4. In various embodiments, laser and treatment parameters (e.g., as shown in FIG. 13) may be selected to optimize efficiency and to treat the tissue without damage. Example treatment patterns are described in more detail in U.S. Patent Publication No. 2017/0319277 and U.S. Patent Publication No. 2018/0325622, both of which are incorporated by reference herein in their entireties and attached as Appendices A and B.

In various embodiments, the laser treatment system 100 can operate in a manner that improves the reduction of caries formation and enhances remineralization. In particular, experiments have been conducted using the laser treatment system 100 to demonstrate this improved performance.

In a first example experiment, a 9.3 μm Solea CO₂ laser (Convergent Dental, Inc., Needham, MA) was used with a beam diameter of 1 mm (measured by 1/e² method) that was collimated at the output of the handpiece over a range of 5 centimeters. The beam was scanned at a repetition rate of 750 Hz using a pair of controllable mirrors (galvanometers) in a pattern with a uniform spacing between centers of adjacent hits of 0.2 mm and a maximum frequency between adjacent hits of 25 Hz. This distribution of hits allowed the energy to be dissipated without accumulation of heat in the pulp. To assist in cooling, a regulated, system-delivered air flow from the handpiece was used on the samples while irradiating with the laser. Laser fluence was varied only by changing the optical pulse duration between 17-27 μs. At 17 μs, the average power is 3.5 W and fluence per pulse is 0.6 J/cm². At 27 μs, the average power is 5.7 W and fluence per pulse is 1.0 J/cm². The distance from the handpiece to the tooth samples was maintained at a set position of 10 mm to further ensure uniform delivery of energy and air to the treatment surface.

To investigate the formation and properties of an acid resistant layer, five sound human enamel samples mounted in acrylic resin and polished to 1 μm diamond grit finish (Therametrics, Inc., Indianapolis, IN) were used. The laser-treated blocks had only been exposed to thymol solution during shipment and were less than 3 months old from extraction. The Solea CO₂ 9.3 μm laser was used with a pulse fluence range of 0.6-1 J/cm². The blocks were serially polished up to 6 μm grit from the side to expose a cross-section of both the laser-irradiated and non-irradiated areas. The original surface that had been laser-irradiated was masked with an acid-resistant tape. The cross-section surface was exposed to 1N HCl for 1 min to erode the underlying ordinary enamel and expose the acid resistant layer, then rinsed thoroughly with water. The blocks were imaged under a 3D digital reflection microscope (Hirox RH-2000) with cross-polarization to capture the subsurface characteristics. The microscope was used to obtain 3D stacks of images over a range of depth of 50 μm.

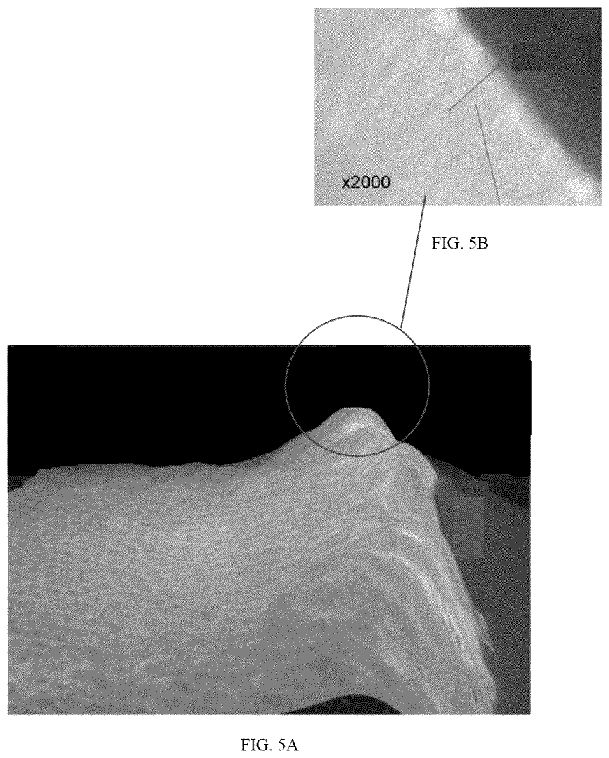

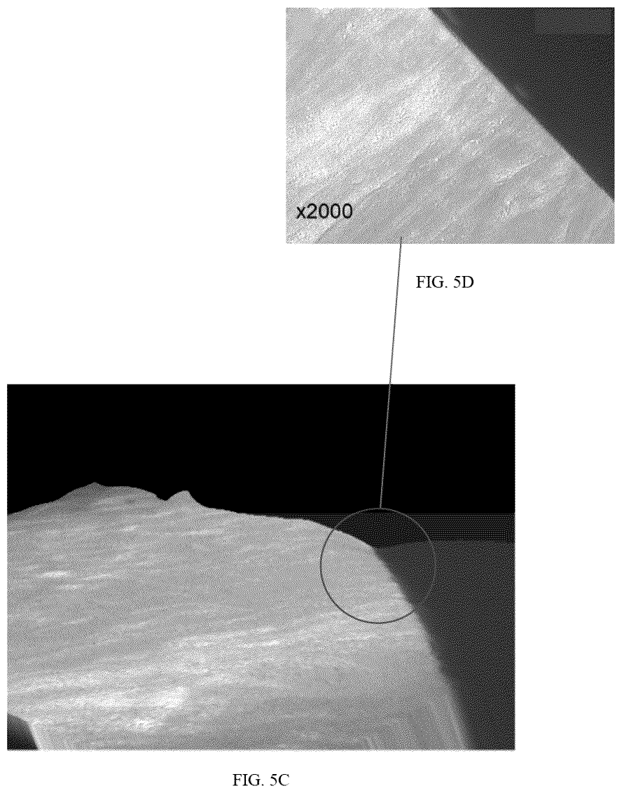

FIG. 5A is a cross-sectioned image of enamel exposed to hydrochloric acid. In this example, an acid-resistant layer in the range of about 15 μm is created by laser-irradiation at a fluence of 0.8 J/cm² and is exhibited as an undissolved protrusion above the underlying enamel that had experienced a rapid dissolution and is out of focus in the microscope image. FIG. 5B shows a cross section of the enamel for the non-irradiated section that dissolved uniformly with no acid-resistant layer observed. This served as a verification that the laser parameters used in the above experiment successfully formed an acid resistant layer on the enamel surface to inhibit demineralization. FIGS. 5C and 5D are 3-dimensional image stacks of the enamel samples that reveal the acid resistant layer as an unetched area near the surface for the laser irradiated sample, and a flat, evenly etched area for the non-irradiated sample.

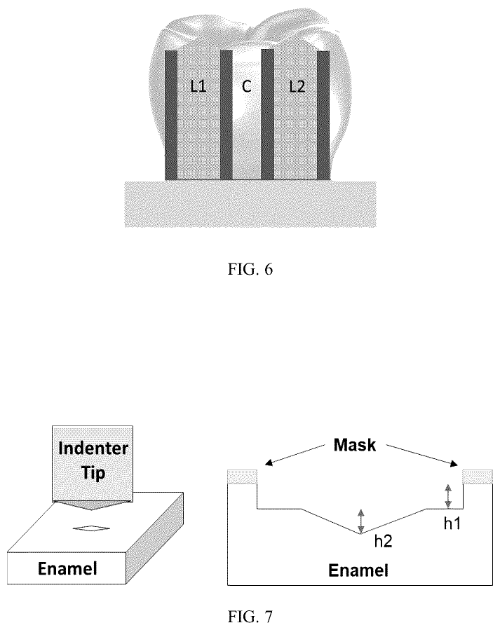

In a second example experiment, to investigate reduction of caries and caries-like legions and the correlation with inhibition of surface mineral loss, seventy-four human molars with no signs of caries or fluorosis and that were less than 3 months old since extraction were obtained and stored only in thymol solution. The molars were mounted in 1″ acrylic cylinders with the full crown exposed (all sides and occlusal surface) (Therametrics, Inc., Indianapolis, IN). The samples were sonicated for 5 minutes in distilled water. The samples were then air dried and split into six groups. Groups 1-3 underwent pH cycling without additional fluoride and groups 4-6 with additional fluoride. Groups 1 and 4 were laser-irradiated at a fluence of 0.6 J/cm², groups 2 and 5 at 0.8 J/cm², and groups 3 and 6 at 1.0 J/cm² on either side of the flattest area (least curvature) of the side of the crown as shown in FIG. 6. Example collected data is shown in Table 1 and Table 2 below (some loss of samples occurred due to polishing damage or other immeasurability of the sample). A non-irradiated control area was maintained in between the laser-irradiated areas. An acid-resistant, quick curing nail polish was used to mask the boundaries between laser-irradiated and non-irradiated regions along the entire height of the molar from the mounted base to the occlusal surface. FIG. 6 depicts a human molar in an acrylic resin mount. Laser-irradiated areas L1 and L2 are depicted, along with the non-irradiated control, C. The stripes are the masked regions over the full height of the exposed tooth.

TABLE 1
pH Cycling Depth Data

| | | Laser | | | | | | |
|---|---|---|---|---|---|---|---|---|
| | Additional | Fluence | Lased | Lased | Non-lased | Non-lased | Lased Reduction | p-value |
| Group | Fluoride | (J/cm²) | ΔZ (std) | ΔZ n | ΔZ (std) | ΔZ n | % ΔZ | (95%) |
| 1 | No | 0.6 | 1025 (253)^{a} | 13 | 1610 (247)^{e} | 13 | 36.3 | p = 0.001 |
| 2 | No | 0.8 | 763 (322)^{ab} | 15 | 1809 (498)^{e} | 15 | 57.8 | p < 0.001 |
| 3 | No | 1.0 | 374 (149)^{bcd} | 5 | 1826 (325)^{e} | 7 | 79.5 | p = 0.002 |
| 4 | Yes | 0.6 | 349 (54.1)^{cd} | 12 | 591 (103)^{b} | 12 | 40.9 | p < 0.001 |
| 5 | Yes | 0.8 | 216 (145)^{d} | 13 | 665 (176)^{b} | 13 | 67.5 | p < 0.001 |
| 6 | Yes | 1.0 | 113 (62.9)^{d} | 7 | 572 (172)^{bc} | 8 | 80.2 | p = 0.01 |

Groups that share a lower-case letter are not significantly different.

TABLE 2
pH Cycling Surface Data

| | | Laser | | | | | | |
|---|---|---|---|---|---|---|---|---|
| | Additional | Fluence | Lased | Lased | Non-lased | Non-lased | Lased Reduction | p-value |
| Group | Fluoride | (J/cm²) | ΔS (std) | ΔS n | ΔS (std) | ΔS n | % ΔS | (95%) |
| 1 | No | 0.6 | 71.8 (17.1)^{ab} | 10 | 124 (26.7)^{e} | 10 | 42.1 | p = 0.002 |
| 2 | No | 0.8 | 55.4 (15.4)^{bc} | 15 | 107 (29.4)^{e} | 15 | 48.2 | p < 0.001 |
| 3 | No | 1.0 | 40.6 (3.6)^{cd} | 5 | 113 (27.6)^{ae} | 5 | 64.1 | p = 0.004 |
| 4 | Yes | 0.6 | 56.9 (15.1)^{bc} | 11 | 92.1 (17.8)^{ae} | 11 | 38.2 | p = 0.006 |
| 5 | Yes | 0.8 | 39.1 (5.4)^{d} | 13 | 100.1 (35.7)^{ae} | 13 | 60.9 | p < 0.001 |
| 6 | Yes | 1.0 | 32.5 (8.4)^{d} | 7 | 88.2 (23.4)^{abe} | 7 | 63.2 | p < 0.001 |

Groups that share a lower-case letter are not significantly different.

Demineralization solution was made in the form of a 75 mM acetate buffer with 2 mM calcium and phosphate, pH balanced to 4.4 using NaOH or HCl where needed. Remineralization solution was made from 0.1 M Tris, 0.8 mM calcium and 2.4 mM phosphate, pH balanced to 7.1. A 9-pH-cycle regimen with the aforementioned solutions was followed similar to the one described in Rechmann P, Rechmann B M T, Groves W H, et al., “Caries inhibition with a CO₂ 9.3 μm laser: An in vitro study,” Lasers Surg Med. 2016;554(February):1-9, doi:10.1002/1sm.22497, with steps of 6 hours in demineralization and 18 hours in remineralization. Approximately half the samples were exposed to a fluoride-toothpaste slurry for 1 min after each step in the cycle, using a 1:3 ratio of Crest cavity protection 1,100 ppm F toothpaste (Proctor and Gamble, Inc.) to distilled water. After 5 cycles, the solutions were replaced with fresh solutions from the same batch. After cycling, samples were stored in distilled water for no more than two weeks until the measurements were performed.

The samples were polished up to 6 at a time using an automated polisher (Metkon Forcipol 1V) with a 600 grit polishing pad until a flat cross-section in the laser-irradiated areas was reached. The samples were individually hand polished with a diamond suspension to remove polish marks for microhardness testing. The samples were serially indented (Matsuzawa Seiki DMH-2) using 25 g loads for 10 s each every 25 μm under the surface, starting at 15 μm from the outer surface until a depth of 200 μm was reached. The volume percent mineral content was calculated at each indentation position using the formula vol %=4.3\sqrt{KHN}+11.3, and ΔZ, a measure of depth mineral loss, was calculated as the area under the curve (with 85% as the normalized level for sound enamel), as described in Stookey G K, Featherstone J D B, Rapozo-Hilo M, et al., “The Featherstone laboratory pH cycling model: A prospective, multi-site validation exercise,” Am J Dent. 2011;24(5):322-328.

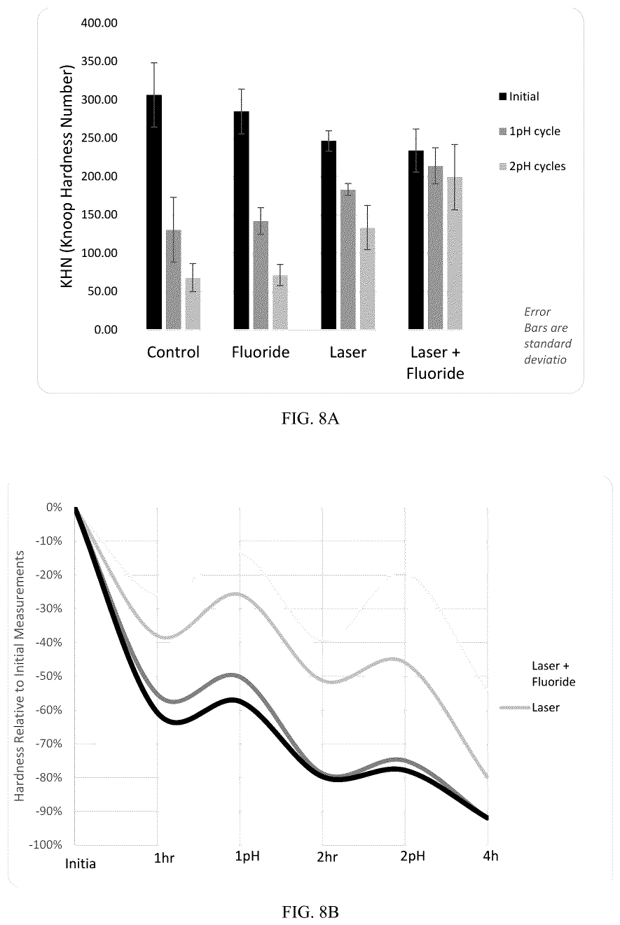

After ΔZ was determined, each sample was turned on its side so that the laser-irradiated areas were facing up. Samples were then polished on this same side until half of the sample had been removed. Occasional loss of sample occurred due to unexpected damage to the surface or mishandling. Using the same indenter, samples were indented 10 times on the laser-irradiated and non-irradiated control surfaces on each region. The symmetry and quality of each indent was checked under the microscope and the lengths of the indents were measured. Surface loss was then measured at the edge of the nail polish using the built-in 3D stage on the microscope. It was determined as the change in height from the edge of the masked surface to the top of the adjacent unmasked enamel surface. Measurements were taken along the entire length of the boundary region at least every 50 μm, obtaining at least 10 measurements for each boundary region. The surface loss and hardness measurements were combined as ΔS=h2/(h1+h2)*vol % as depicted in FIG. 7, where vol % is calculated from the indents as described above. FIG. 7 depicts a diagram of creating an indent on enamel (left) and a cross-sectional view of the indent site (right). For the calculation of ΔS, h1=surface loss, h2=microhardness indent height, and vol % is calculated from the size of the indent on the surface. ΔS is a representation of the amount of mineral loss on the surface relating to a slow surface mineral loss; whereas, ΔZ is a measure of mineral loss in depth relating to a caries-like formation under the surface. Data were analyzed on a log-scale using Welch's ANOVA and post-hoc Games-Howell tests between groups. FIG. 8A is an example chart showing knoop hardness number measurements of various tissue surfaces subject to different pH cycle tests, according to various embodiments. FIG. 8B is an example plot depicting changes in hardness of various tissue surfaces subject to different pH cycle tests, according to various embodiments.

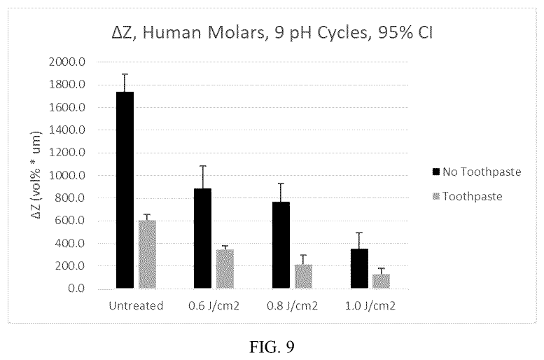

ΔZ values, shown in Table 1 and FIG. 9, provide a measure of caries-like lesion formation and serve as a metric for comparing treatments with different laser settings. For the range of fluences used, there were no observed superficial structural changes. However, there were occasional signs of minor fracture-like structural changes under the surface for the areas irradiated with 1.0 J/cm², possibly related to subsurface thermal fracturing.

Welch's ANOVA applied to the data showed that there were significant differences between the groups (F_{11,40}=81.0, p<0.001). The average reduction in ΔZ from use of additional fluoride (groups 4-6) without laser-irradiation was —65% (p<0.001). Reductions in ΔZ from laser irradiation alone were observed (see groups 1-3 in Table 1), which indicates that an effective remineralization can and does occur, with or without the presence of additional fluoride. Post-hoc Games-Howell tests showed that the combination treatment of laser-irradiation and additional fluoride provided the most significant benefit in reducing ΔZ for each of the laser fluences used (p<0.01 for all). Although pH-cycling with fluoride toothpaste alone revealed a benefit in caries inhibition, application of fluoride toothpaste in an area that had been irradiated as described herein resulted in the most significant and unexpectedly high reduction in caries formation, with as high as a ˜92% (p=0.001) reduction in ΔZ compared to the untreated control areas.

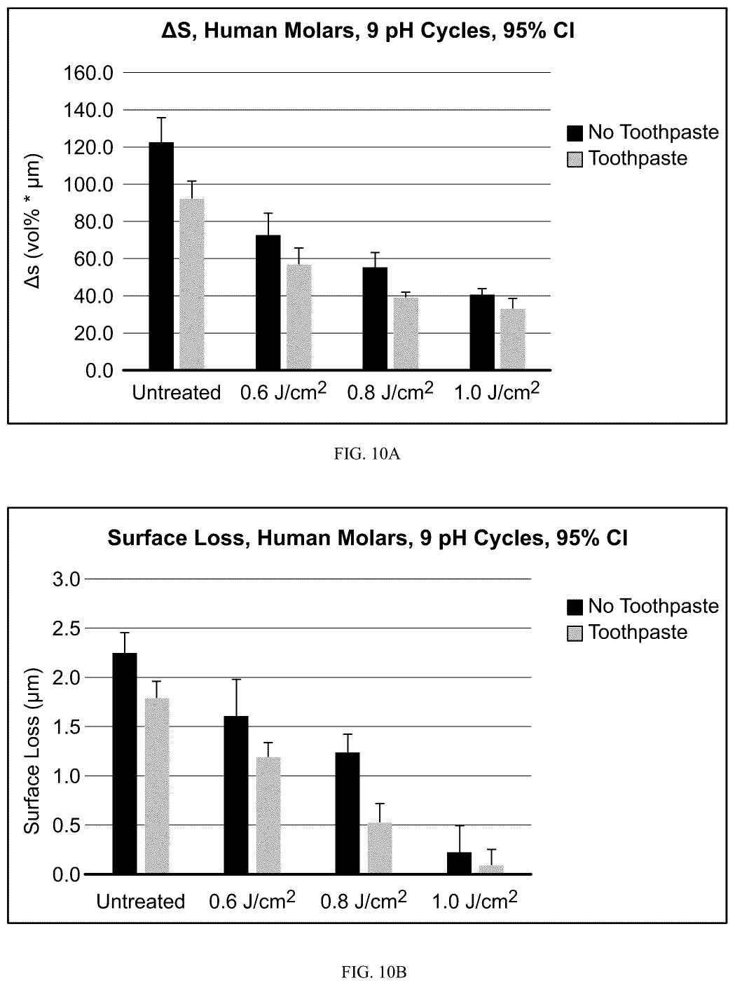

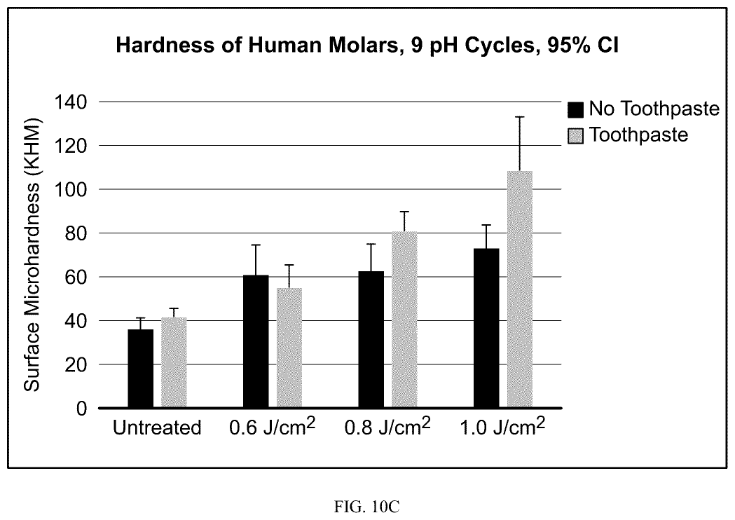

Table 2 and FIGS. 10A-10C show a combination of the surface metrics, in the form of ΔS. As with ΔZ, Welch's ANOVA applied to ΔS values showed that there were significant differences between groups (F_{11,36}=39.5, p<0.001). ANOVA showed no differences within the non-irradiated groups without additional fluoride (F_{2 ,12}=1.16, p>0.05) or with additional fluoride (F_{2,16}=0.22, p>0.05). The general trends for ΔS were similar to those observed for ΔZ in relation to laser fluence. ΔS values showed no significance from fluoride use alone relative to untreated controls through post-hoc Games-Howell tests (p>0.05). However, comparing all data without additional fluoride to that with additional fluoride using Student's t-test, a significant reduction of ΔS by ˜18% (p<0.001) was found. ΔS revealed that laser-irradiation alone at 9.3 μm inhibits surface mineral loss by as much as ˜64% (p=0.004). Furthermore, a combination of 1 J/cm² μser-irradiation coupled with fluoride application from toothpaste showed an unexpectedly high reduction in surface mineral loss of ˜72% (p<0.001) compared to the untreated control.

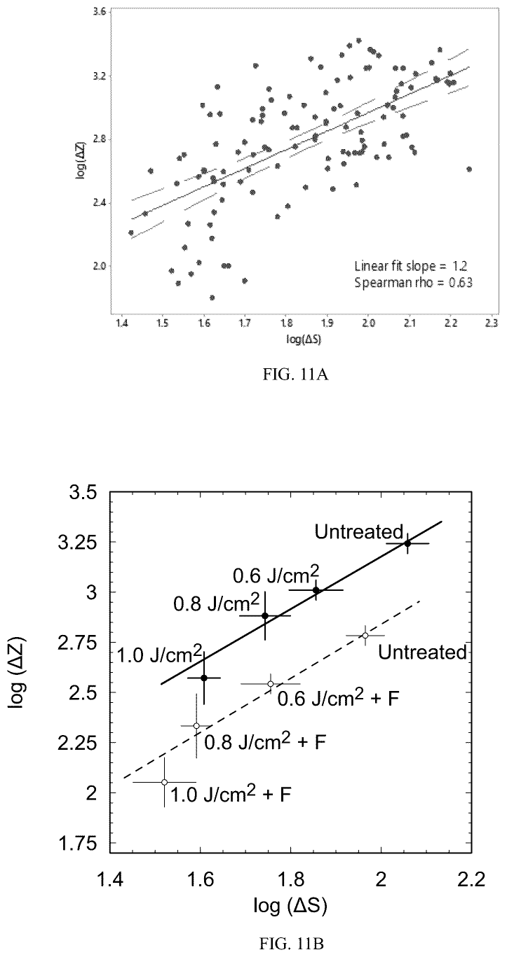

FIG. 11A is a plot of ΔZ values as a function of ΔS values for the measurements collected in the above-described experiment. ΔZ and ΔS values followed a log-normal distribution, and a linear trend between them was observed with a Spearman rho of 0.63 (p<0.001). FIG. 11B shows linear regression fits (on a log scale) when the data points were averaged according to treatment group. Since the control and toothpaste-cycled sets were different in methodology, they are treated as distinct sets. The linear fits for these two sets have very similar slopes, ˜1.3, both with an R²>0.95. This discovery provides two very important pieces of information. One is that the overall effect of fluoride on caries and acid resistance can be quantified by the vertical shift in the curve, revealing that fluoride provides a 50-60% inhibition in caries formation/acid penetration. The other is that the benefit of the laser can be quantified for each pulse fluence. Averaged both with and without additional fluoride, ΔZ and ΔS were remarkably improved by ˜39%, 59%, and 72% after laser-irradiation of 0.6, 0.8 and 1.0 J/cm², respectively, compared to areas without laser-irradiation.

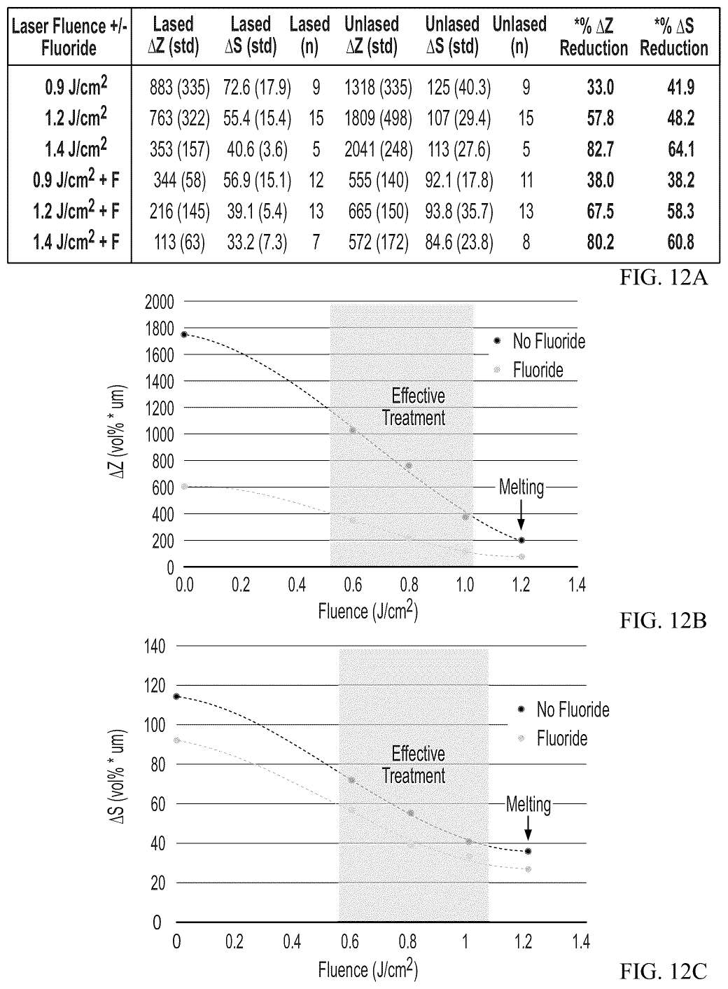

FIG. 12A provides example data, including ΔZ and ΔS measurements, for tissues lased at various fluences and with and without the application of fluoride. FIG. 12B is a graph of measured ΔZ values for various fluences for treatments combined with fluoride and without fluoride, as well as an indication of an example effective treatment zone. FIG. 12C is a graph of measured ΔS values for various fluences for treatments combined with fluoride and without fluoride, as well as an indication of an example effective treatment zone.

In various embodiments, laser irradiation at 9.3 μm creates an acid-resistant form of hydroxyapatite that occurs at temperatures in which a large amount of the carbonate groups in the carbonated-hydroxyapatite mineral of the dental enamel are removed. This can allow a crystallization of fluoride-containing hydroxyapatite in these weak areas to occur, thereby reducing the penetration of acid under the resistant layer. Other organic components, which largely exist as a glue-like network holding adjacent rods together, may also be removed near the surface during irradiation. This surface change has been described previously as a “glazing” of the surface.

In various embodiments, the benefit of laser-irradiation under the laser conditions tested, even without the use of fluoride, is significant. The softening of the surface and formation of an underlying lesion can be slowed significantly. This may be an indication that a crystallization of the “weakened” sites occurs from dissolved minerals (primarily calcium and phosphate), despite an initial acid exposure. Due to this remineralization effect, the benefit of laser-induced acid resistance can, outweigh the risks associated with structural changes to the surface. In some embodiments, the introduction of fluoride in the cycling process can enhance the acid resistant properties of the layer, which may be due at least in part to the inherent resistance of fluorapatite to dissolution. The laser treated area may encourage an uptake of fluoride by the surface, together with calcium and phosphate, resulting in the observed beneficial effect of the pair of treatments in combination. As described below, in some embodiments that application of fluoride can be performed with delivery of the coolant with the laser treatment system 100. However, the invention also contemplates delivery of fluoride from other sources, e.g., separate from delivery through the laser treatment system 100, either as part of a dental office visit or elsewhere (e.g., brushing teeth or using mouthwash at home).

The inventions described herein demonstrate a direct correlation between the surface mineral loss (ΔS) and the size of a carious lesion (ΔZ). In some embodiments, one explanation for this is that a complete remineralization back to the original density may not be possible, as the crystallization is random and may introduce insufficiently packed crystals. Through laser irradiation of the surface, the softening of the enamel from acid exposure may be significantly reduced, which was evident in both measures of surface (erosion) or depth (caries) mineral loss. The resistance to acid may be further enhanced by use of a high-concentration fluoride application, such as prescription mouthwash or varnish, which would also help to quickly remineralize any weak sites with fluorapatite, possibly even before a patient leaves the treatment clinic, as the laser-irradiated area is capable of increasing the rate of fluoride uptake. In this work, erosion and caries resistance may be enhanced by around 50-60% using fluoride-containing toothpaste and can be increased by a further 40-80% using a laser treatment with a 9.3 μm CO₂ laser.

As used herein, the term “working range” means the distance along the length of the laser beam at which the laser beam has a fluence capable of treating the tissue (e.g., removing carbonate). Conventional devices have a relatively short working range, typically focused tightly around a focal point of the laser beam based on a desire to not waste any energy along the length of the laser. The laser treatment system of the present invention can, in some embodiments, tolerate a longer working range, so as to enable an operator to move their hand (and, correspondingly, the laser beam), while still treating the treatment area effectively. In other words, in certain embodiments, the amount of energy delivered to the target tissue does not change over a relatively long distance along the axis between the exit orifice 8 and the hard tissue (e.g., more than 0.5 cm, more than 1 cm, more than 1.5 cm, more than 2 cm, more than 3 cm, more than 4 cm, etc.) to accommodate for hand movements, variability in the user's holding of the handpiece standoff and other human factors. The concept of a working range is described in more detail with reference to the phrase “depth of treatment” (which can be interchanged with “working range”) in U.S. Patent Publication No. 2016/0143703, which is incorporated by reference herein in its entirety and attached as Appendix C.

In some embodiments, the hand piece 1 can also be adapted to transmit fluids to the treatment region, e.g., cooling fluids and/or fluoride-based fluids. The fluids can be transported using any known technique, e.g., through fluid tubing 5 that run along the handpiece 1 and bypass the optical cartridge 2. The cooling fluids can be useful to minimize and avoid excessive heating of the tissue. As discussed above, the fluoride based fluids may result in improved and more effective treatment. In some cases, both cooling and fluoride based fluids can be delivered through the same tubings 5. In other cases, the hand piece 1 can include separate tubing for each of the cooling fluid and fluoride-based fluid. Other desirable fluids may also be transported through the hand piece 1.

In certain embodiments, the laser beam is accompanied with a marking beam (e.g., green in color) that serves as guidance of the location of the laser beam on the target tissue. In some instances, the irradiation of the laser may occur in a pattern. A visual or sonar feedback can optionally be integrated within the system 100 to indicate to the user the need to move to a new target area. A visual feedback indicating a new target area can include a stationary guidance beam (e.g., a green point projected on the tissue). For example, while the tissue is being exposed to the laser, a pattern can be displayed on the tissue. When enough energy has been delivered, the laser can stop scanning and a point object can being projected on the target tissue. Alternatively, a sonar feedback can be provided to indicate a pattern and/or amount of energy delivery.

FIG. 13 is a chart including example laser and operation parameters for the laser treatment system 100. The parameters may be designed to have a desired outcome efficiency to remove carbonate without damaging the treatment surface material. In some embodiments, the laser beam may be spatially scanned to provide different pulse energy at different locations, as will be appreciated by those skilled in the art.

Each numerical value presented herein is contemplated to represent a minimum value or a maximum value in a range for a corresponding parameter. Accordingly, when added to the claims, the numerical value provides express support for claiming the range, which may lie above or below the numerical value, in accordance with the teachings herein. Every value between the minimum value and the maximum value within each numerical range presented herein (including the low, nominal, and high values shown in the chart shown in FIG. 13), is contemplated and expressly supported herein, subject to the number of significant digits expressed in each particular range.

Having described herein illustrative embodiments of the present invention, persons of ordinary skill in the art will appreciate various other features and advantages of the invention apart from those specifically described above. It should therefore be understood that the foregoing is only illustrative of the principles of the invention, and that various modifications and additions, as well as all combinations and permutations of the various elements and components recited herein, can be made by those skilled in the art without departing from the spirit and scope of the invention. Accordingly, the appended claims shall not be limited by the particular features that have been shown and described but shall be construed also to cover any obvious modifications and equivalents thereof.

1.-19. (canceled)

20. A method for treating a hard dental tissue, the method comprising the steps of:
generating a laser beam using a laser source;
directing the laser beam to a treatment surface of the hard dental tissue using an optic in optical communication with the laser source; and
controlling the laser source and the optic using a controller to deliver the laser beam to the treatment surface to treat an area of the hard dental tissue at a rate in a range from 10 cm²/min to 20 cm²/min with a fluence in a range from 0.4 J/cm² up to 1.2 J/cm² to:
remove at least some carbonate from the treatment surface to generate an acid resistant surface without damaging the hard dental tissue; thereafter determining a depth mineral loss (ΔZ) value of the hard dental tissue; and
confirming the ΔZ value of the hard dental tissue [[by]]is at least 10% relative to untreated hard dental tissue.

21. The method of claim 20, wherein the laser source comprises a CO₂ laser source.

22. The method of claim 21, wherein the laser beam comprises a wavelength in a range from 9 μm to 11 μm.

23. (canceled)

24. The method of claim 20, wherein the optic comprises at least one of a galvanometer and a turning mirror.

25. The method of claim 20, wherein the controller is further adapted to control the laser source to deliver the laser beam to the treatment surface in a series of pulses.

26. The method of claim 25, wherein each pulse in the series of pulses comprises a pulse energy in a range from 0.1 mJ to 50 mJ.

27. The method of claim 25, wherein each pulse in the series of pulses comprises a pulse duration in a range from 1 ilsec to 100 ilsec.

28. The method of claim 25, wherein the series of pulses comprises a repetition rate in a range from 0.05 Hz to 10 Hz.

29. The method of claim 25, wherein the series of pulses comprises a duty cycle in arrange from 0.1 to 10.

30. The method of claim 25, wherein the controller is adapted to deliver the series of pulses to the treatment surface in a pattern.

31. The method of claim 30, wherein the pattern comprises a diameter in a range from 1 mm to 5 mm.

32. The method of claim 30, wherein the pattern comprises a number of locations in a range from 1 to 1,000.

33. The method of claim 32, wherein the pattern comprises 217 locations.

34. The method of claim 32, wherein a spacing between each location in the pattern is in a range from 0.1 mm to 0.5 mm.

35. The method of claim 20, wherein the controlling step further comprises using the controller to control the laser source to deliver the laser beam to the treatment surface to, when combined with a fluoride treatment, reduce a ΔZ value of the dental tissue by at least 20% relative to hard dental tissue subject to the fluoride treatment.

36. The method of claim 35, further comprising the step of delivering the fluoride treatment to the treatment surface.

37. The method of claim 20, wherein the controlling step further comprises using the controller to control the laser source to deliver the laser beam to the treatment surface to reduce a ΔS value of the dental tissue by at least 68% relative to untreated dental tissue.

38. The method of claim 20, wherein the controlling step further comprises using the controller to control the laser source to deliver the laser beam to the treatment surface to, when combined with a fluoride treatment, reduce a ΔS value of the dental tissue by at least 18% relative to hard dental tissue subject to the fluoride treatment.
